(19) United States
(12) Patent Application Publication
(10) Pub. No.: US 2025/0381195 A1
(43) Pub. Date: Dec. 18, 2025

(54) Methods of Treating Diabetic Macular Edema

(71) Applicant: UNM Rainforest Innovations, Albuquerque, NM (US)

(72) Inventors: Arup Das, Albuquerque, NM (US); Finny M. Monickaraj, Albuquerque, NM (US)

(21) Appl. No.: 19/208,897

(22) Filed: May 15, 2025

Related U.S. Application Data
(60) Provisional application No. 63/660,599, filed on Jun. 17, 2024.

(51) Int. Cl. A61K 31/55; A61K 45/06

(52) U.S. Cl. CPC A61K 31/55; A61K 45/06

(57) ABSTRACT

The present invention is directed to methods for treating diabetic macular edema (DME), proliferative diabetic retinopathy (PDR), wet age-related macular degeneration (AMD) and/or retinal vein occlusion (RVO) in a patient or subject in need comprising administering an effective amount of at least one anti-DME agent selected from the group consisting of desipramine, protriptyline, cyclosporin A, crisaborole, empagliflozin, nitroxolone, suprofen, sulfisoxazole, methapyrilene, phentolamine, naphazoline or a pharmaceutically acceptable salt thereof, preferably at least one agent selected from the group consisting of desipramine, nitroxolone, methapyriline, phentolamine, napthazoline and their pharmaceutically acceptable salts.

### RELATED APPLICATIONS

This application claims the benefit of priority of United States provisional application U.S. Ser. No. 63/660,599, filed Jun. 17, 2024 of identical title, the entire contents of which application is incorporated by reference in its entirety herein.

### FIELD OF THE INVENTION

The present invention is directed to methods for treating diabetic macular edema (DME), proliferative diabetic retinopathy (PDR), wet (exudative) age-related macular degeneration (wet AMD) and/or retinal vein occlusion (RVO) in a patient or subject in need comprising administering an effective amount of at least one anti-DME agent selected from the group consisting of desipramine, protriptyline, cyclosporin A, crisaborole, empagliflozin, nitroxolone, suprofen, sulfisoxazole, methapyrilene, phentolamine, naphazoline or a pharmaceutically acceptable salt thereof, preferably at least one agent selected from the group consisting of desipramine, nitroxolone, methapyriline, phentolamine, napthazoline and their pharmaceutically acceptable salts. In embodiments, a patient or subject is first diagnosed or identified with diabetic macular edema, proliferative diabetic retinopathy (PDR), wet (exudative) age-related macular degeneration (wet AMD) and/retinal vein occlusion (RVO) or for being at risk for any one or more of these disease states/conditions and subsequently is treated with an anti-DME agent as otherwise disclosed herein (desipramine, protriptyline, cyclosporin A, crisaborole, empagliflozin, nitroxolone, suprofen, sulfisoxazole, methapyrilene, phentolamine, naphazoline or a pharmaceutically acceptable salt thereof, preferably at least one agent selected from the group consisting of desipramine, nitroxolone, methapyriline, phentolamine, napthazoline and their pharmaceutically acceptable salts often desipramine) to treat, inhibit or reduce the likelihood of DME, PDR, wet AMD and/or RVO in the patient or subject. In preferred embodiments, the anti-DME agent is at least one agent selected from the group consisting of desipramine, nitroxolone, methapyriline, phentolamine, napthazoline and their pharmaceutically acceptable salts. Often, the anti-DME agent used to treat these disease states or conditions is nitroxolone, methapyriline, phentolamine, napthazoline and their pharmaceutically acceptable salts or a mixture thereof. In embodiments, the agent is despiramine. In embodiments, the administration of one or more of the above-described agents to a patient or subject in need is preceded by a diagnosis or identification of the patient or subject with one or more of these disease states and/or conditions, especially diabetic macular edema or for being at risk of diabetic macular edema. In embodiments, one or more anti-DME agents as described above is co-administered in combination with an anti-VEGF agent such as ranibizumab, aflibercept, bevacizumab, sorafenib, dasatinib, sunitinib, nilotinib and pazopanib, a corticosteroid such as dexamethasone, bethamethasone, prednisone, prednisolone, methylprednisolone and triamcinolone and/or an insulin-like growth factor receptor inhibitor such as figitumumab (CP-751871), ganitumab (AMG-479), cixutumumab (IMC-A12), dalotuzumab (MK-0648), R1507, Sch-717454, BMS-754807, linsitinib (OSI-906), MEDI-573 and BI836845) for the treatment of DME, PDR, we AMD and/or RVO with neovascularization. Pharmaceutical compositions comprising these agents in combination are also contemplated by the present invention as described herein.

### BACKGROUND AND OVERVIEW OF THE INVENTION

Diabetic macular edema (DME) is a severe complication of diabetic retinopathy (DR) that occurs often as a result of inadequately treated diabetes mellitus (DM) and has overtaken proliferative diabetic retinopathy (PDR) as the most common cause of vision impairment in individuals with diabetes mellitus type II. In recent epidemiologic studies, approximately 30% of patients worldwide with DM were found to have vision-threatening DR; and in the United States, 3.8% of patients were found to have DME.

Present therapies which are used to treat DME, especially VEGF inhibitors, are often only partially successful in approximately 30% of patients, so the discovery of additional therapies for the treatment of this disease state represents a critical concern. The present invention seeks to address the critical need in the art for treatments for DME in the patient population.

### BRIEF DESCRIPTION OF THE INVENTION

In an embodiment, the present invention is directed to methods for treating, inhibiting or reducing the likelihood of diabetic macular edema (DME), proliferative diabetic retinopathy (PDR), wet (exudative) age-related macular degeneration (wet AMD) and/retinal vein occlusion (RVO) in a patient or subject comprising administering an effective amount of at least one anti-DME agent selected from the group consisting of desipramine, protriptyline, cyclosporin A, crisaborole, empagliflozin, nitroxolone, suprofen, sulfisoxazole, methapyrilene, phentolamine, naphazoline or a pharmaceutically acceptable salt thereof, preferably at least one agent selected from the group consisting of desipramine, nitroxolone, methapyriline, phentolamine, napthazoline and their pharmaceutically acceptable salts. In certain embodiments, a patient or subject is first diagnosed or identified with DME, PDR, wet AMD and/or RVO or for being at risk for one or more of these disease states and/or conditions and is treated with an agent as otherwise disclosed herein to treat, inhibit or reduce the likelihood of DME, PDR, wet AMD and/or RVO in the patient or subject. In embodiments, one or more the agents described above is co-administered in combination with an effective amount of anti-VEGF agent such as ranibizumab, aflibercept, bevacizumab, pegaptanib, faricimab (faricimab-svoa), sorafenib, dasatinib, sunitinib, nilotinib and pazopanib, a corticosteroid such as dexamethasone, fluocinolone acetonide, bethamethasone, prednisone, prednisolone, methylprednisolone and triamcinolone and/or an insulin-like growth factor receptor inhibitor such as figitumumab (CP-751871), ganitumab (AMG-479), cixutumumab (IMC-A12), dalotuzumab (MK-0648), R1507, Sch-717454, BMS-754807, linsitinib (OSI-906), MEDI-573 and BI836845) for the treatment or inhibition of DME, PDR, wet AMD and/or RVO.

In an embodiment, the present invention is directed to pharmaceutical compositions comprising combinations of an effective amount of at least one anti-DME agent as set forth above and at least one additional anti-DME agent (e.g. an anti-VEGF agent, a corticosteroid and/or an insulin-like growth factor receptor inhibitor as described herein) optionally in combination with at least one pharmaceutically acceptable carrier, additive and/or excipient.

### BRIEF DESCRIPTION OF THE FIGURES

FIG. 1 shows Phenotypes of Diabetic Macular Edema (DME): A. Non-center involving DME, also called the Focal type, macular edema does not involve the center of the macula, not vision threatening; usually these patients may be observed or rarely treated with focal/grid laser therapy. B. Center-involving DME, macular edema involves the center of the macula, vision threatening; these patients may be observed if vision is 20/20-20/25, and if worse, may be treated with intravitreal anti-VEGF injections.

FIG. 2 addresses the question is there a genetic factor that determines “Good” vs. “Poor” Responders to anti-VEGF treatment? Good Responder: The left panel shows the OCT images of both eyes of a DME patient treated with one intravitreal anti-VEGF injection, and central retinal thickness is normalized with vision improvement, Upper: Before the injection, Lower: After the injection. Poor Responder: The right panel shows the OCT images of both eyes of a DME patient treated with six monthly intravitreal injections of anti-VEGF drugs, and still the central retinal thickness did not change significantly. Upper: Before the injection. Lower: After 6 monthly injections.

FIG. 3 shows A. Diagram showing how ECIS (Endothelial Cell-Substrate Impedance Sensing) works (Courtesy Applied Biophysics): This technique measures the electrical resistance between endothelial cells in culture. The 250-um diameter gold electrode measures the ion current in the cell layer and measures the resistance and how the barrier function is altered with VEGF and drug treatment. B. Drug Library for repurposed drugs. The Prestwick Chemical Library consists of 1500 off-patent compounds approved by the FDA, EMA and other agencies with diverse chemical and pharmacological characters. All these drugs fall under the WHO Anatomical Therapeutic Chemical (ATC) Classification 5 which contains all chemical substances which are specifically designed to increase the potency of the drug with a mean molecular weight of 383. More than half of the drugs within the library are dedicated to CNS, cardiovascular, metabolism and infectious diseases.

FIG. 4 shows a list of repurposed drugs that show decreased permeability. The Table shows different repurposed drugs that have been found to have decreased permeability in the cell culture model using the ECIS system.

FIG. 5 shows the top five drugs that have been tested and shown to have decreased permeability function in the in vitro model. It shows the mechanism of action (MOA), in vivo concentration, market brand name.

FIG. 6 shows in vivo studies proposed for testing these five drugs in vivo in an animal model of diabetes. The endpoint will be measurement of extravascular albumin, cell junction proteins, immune cell infiltration using the FACS method.

FIG. 7 shows the ECIS Results on 1500 drugs from the Drug Library showing changes in resistance of EC after addition of drugs (Desipramine HCl, Protriptyline HCl, Cyclosporin A, Crisaborole, Empaglifozin and Nitroxolone). Each of six drugs showed significant increase in resistance of EC layer after addition of VEGF (50 ng) in the endothelial cell layer followed by the addition of the tested drug (10 uM).

FIG. 8 shows the ECIS Results of Other drugs in the ECIS system showing increased resistance of the EC layer after addition of the drugs (Suprofen, Sulfisoxazole, Methapyrilene, Phentolamine, Naphazoline HCl).

### DETAILED DESCRIPTION OF THE INVENTION

It is noted that, as used in this specification and the appended claims, the singular forms “a,” “an,” and “the,” include plural referents unless expressly and unequivocally limited to one referent. Thus, for example, reference to “a compound” includes two or more different compounds. As used herein, the term “include” and its grammatical variants are intended to be non-limiting, such that recitation of items in a list is not to the exclusion of other like items that can be substituted or other items that can be added to the listed items.

The term “compound” or “agent”, as used herein, unless otherwise indicated, refers to any specific chemical compound disclosed herein and includes tautomers, regioisomers, geometric isomers as applicable, and also where applicable, optical isomers (e.g. enantiomers) and isotopomers thereof, as well as pharmaceutically acceptable salts thereof. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds as well as diastereomers and epimers, where applicable in context. The term also refers, in context to prodrug forms of compounds which have been modified to facilitate the administration and delivery of compounds to a site of activity.

The term “patient” or “subject” is used throughout the specification within context to describe an animal, generally a mammal, including a domesticated mammal including a farm animal (dog, cat, horse, cow, pig, sheep, goat, etc.) and preferably a human, to whom treatment, including prophylactic treatment (prophylaxis), with the methods and compositions according to the present invention is provided. For treatment of those conditions or disease states which are specific for a specific animal such as a human patient, the term patient refers to that specific animal, often a human. In embodiments, the patient is a patient who has been diagnosed specifically with diabetic macular edema. In embodiments, the patient has been diagnosed with proliferative diabetic retinopathy. In embodiments, the patient with diabetic macular edema also is diagnosed with or has proliferative diabetic retinopathy. In embodiments, the patient with diabetic macular edema is free from proliferative diabetic retinopathy. In embodiments, the patient or subject with proliferative diabetic retinopathy is free from diabetic macular edema. In embodiments, the patient or subject may be diagnosed as having one or more or being at risk for one or more of DME, PDR, wet AMD and/or RVO. In embodiments, the patient or subject may be diagnosed with only one of DME, PDR, wet AMD and/or RVO or be at risk for only one of DME, PDR, wet AMD and/or RVO and be free from the other disease states and/or conditions as indicated.

The terms “effective”, “therapeutically effective” or “pharmaceutically effective” are used herein, unless otherwise indicated, to describe an amount of a compound or composition which, in context, is used to produce or affect an intended therapeutic result, usually the inhibition, amelioration and/or prevention (reduction in the likelihood) of diabetic macular edema, proliferative diabetic retinopathy, wet (exudative) age-related macular degeneration (wet AMD) and/or retinal vein occlusion (RVO) within the context of a particular treatment or alternatively, the effect of a bioactive agent which is coadministered with the anti-DME agent in the treatment or prophylaxis (especially reducing the likelihood) of disease.

The terms “treat”, “treating”, and “treatment”, etc., as used herein, refer to any action providing a benefit to a patient at risk for or afflicted by diabetic macular edema (DME), proliferative diabetic retinopathy (PDR), wet (exudative) age-related macular degeneration (wet AMD) and/or retinal vein occlusion (RVO) or a condition as otherwise described herein. The benefit may be in curing the disease state or condition, inhibition its progression, or ameliorating, lessening or suppressing one or more symptoms of diabetic macular edema. Treatment, as used herein, encompasses both prophylactic and therapeutic treatment. Prophylactic, when used, refers to “reducing the likelihood” of a disease state, condition or symptom associated with same occurring.

The term “diabetic macular edema” or “DME” is used to describe a condition which occurs secondary to diabetes (diabetes mellitus type I or II, especially type II diabetes mellitus) in a patient or subject in which the accumulation of excess fluid in the extracellular space within the retina in the macular area, typically in the inner nuclear, outer plexiform, Henle's fiber layer, and subretinal space. Chronic hyperglycemia-related accumulation of glycated products can disrupt the blood retinal barrier (BRB) characterized by endothelial cell junction breakdown and pericyte loss. The inner BRB is composed of endothelial cells in the retinal capillaries, while the outer BRB is composed of retinal pigment epithelium (RPE) cells. Altered BRB leads to interstitial fluid accumulation within and underneath the retina through leakage of molecules dependent on intact cell to cell junctions. Evidence also shows that DME has an inflammatory component to the disease, with several chemokines and cytokines involved in its development. These factors include vascular endothelial growth factor (VEGF), interleukins (ILs), matrix metalloproteinases (MMPs), and tumor necrosis factor (TNF). Upregulation of multiple pathways leads to increased inflammation, oxidative stress, and vascular dysfunction. There are also significant changes in the neurovascular unit, altering the homeostasis between astrocytes, ganglion cells, Müller cells, retinal vascular endothelial cells, and amacrine cells. Retinal vascular permeability changes also involve the kallikrein-kinin system, which induces vasorelaxation via bradykinin and nitric oxide.

DME can develop at any stage of diabetic retinopathy (DR), from mild nonproliferative diabetic retinopathy (NPDR) to proliferative diabetic retinopathy (PDR), but is more frequent as the severity of DR increases. DME threatening or at the fovea is more likely to result in blurred vision and metamorphopsia. When the DME involves or threatens the fovea, the risk of moderate visual loss (MVL, defined as a three-line or more decrease of visual acuity, equivalent to a doubling of the visual angle) over 3 years in the Early Treatment of Diabetic Retinopathy Study (ETDRS) can be substantial (i.e., 24%) without treatment. The disease course is variable, with some eyes having chronic persistent DME spanning several years, while other eyes have rapid spontaneous resolution, although the risk of recurrence is always present.

“Proliferative diabetic retinopathy” or (PDR) is the advanced stage of diabetic retinopathy. In proliferative diabetic retinopathy, new blood vessels grow in the retina, the light-sensing layer at the back of the eye. This serious condition can lead to vision loss. Diabetic retinopathy can also cause some blood vessels to shut down. When there isn't enough blood supply to the retina, a signal is sent out that spurs the development of new blood vessels. But these blood vessels are abnormal and prone to leaking. This advanced stage, where such blood vessels develop, is known as PDR. The bleeding from these abnormal new vessels can lead to scarring on the retina. Ultimately, the scarring can lead to retinal detachment in which the retina separates from the back of the eye. In turn, this can lead to vision loss. Abnormal new vessels can also grow in the front of the eye and block the normal drainage of fluid, leading to elevated eye pressure (neovascular glaucoma). Elevated eye pressure can then cause permanent damage to the optic nerve, which can also contribute to permanent vision loss.

“Wet (exudative) age-related macular degeneration”, “wet AMD” or “wet ARMD” is the most common cause of visual impairment among elderly patients in developed countries. Approximately 10% of patients with dry ARMD will develop choroidal neovascularization (CNV), which is the hallmark of wet ARMD. Vascular endothelial growth factor (VEGF) drives the development of CNV, which may lead to bleeding under the retina, detachment or atrophy of the retinal pigment epithelium (RPE), or sub-retinal or sub-RPE fluid accumulation with associated vision loss.

Wet AMD is differentiated from early or dry ARMD by the presence of choroidal neovascularization (CNV), where new blood vessels from the choroid penetrate through the Bruch membrane and proliferate either between Bruch's membrane and the RPE or in the subretinal space. Various factors contribute to the development of CNV and vision loss in patients with wet ARMD. These factors include:
VEGF accumulation, particularly the VEGF (165) isoform;
Growth of new blood vessels with the proliferation of fibrous tissue;
Leakage of proteins and lipids from the new vessels;
Hemorrhage from the fragile new vessels;
Fibrovascular scar formation, with the death of the neurosensory retina and vision loss.

Wet AMD is evaluated/diagnosed in a patient using Optical Coherence Tomography (OCT), OCT angiography (OCT-A), Fluorescein Angiography (FA) and/or Indocyanine Green Angiography (IGA).

“Retinal vein occlusion” or “RVO” is an important cause of visual loss among older adults throughout the world. RVO is the second most common cause of vision loss from retinal vascular disease, following diabetic retinopathy. Until the present invention, and despite many proposed interventions, there were no treatments proven to reopen occluded retinal veins. Management was principally directed at secondary complications of RVO that affect vision, including macular edema, retinal neovascularization, and anterior segment neovascularization. Effective treatment for macular capillary nonperfusion, a fourth cause of visual loss in RVO, has now been made available.

The term “co-administration” or “combination therapy” is used to describe a therapy in which at least two active compounds in effective amounts are used to treat diabetic macular edema, PDR, wet AMD and/or RVO as otherwise described herein, either at the same time or within dosing or administration schedules defined further herein or ascertainable by those of ordinary skill in the art. Although the term co-administration preferably includes the administration of at least two active compounds to the patient at the same time in one or more parts, it is not necessary that the compounds be administered to the patient at the same time, although effective amounts of the individual compounds will be present in the patient at the same time. In addition, in certain embodiments, co-administration will refer to the fact that two or more compounds are administered at significantly different times, but the effects of the two compounds are present at the same time.

Compounds used in the methods of treatment of the present invention may be used in pharmaceutical compositions having biological/pharmacological activity for the treatment of, for example, diabetic macular edema (DME), proliferative diabetic retinopathy (PDR), wet (exudative) age-related macular degeneration (wet AMD) and/or retinal vein occlusion (RVO) and/or disease states or conditions/symptoms which may appear or occur secondary to these conditions. These compositions comprise an effective amount of any one or more of the compounds disclosed hereinabove, optionally in combination with a pharmaceutically acceptable additive, carrier or excipient.

The term “anti-DME” agent is used to describe a compound/agent which shows an inhibitory or other effect favorable to the treatment or prevention of DME, PDR, wet AMD and/or RVO in patients or subjects. These compounds include, for example, desipramine, protriptyline, cyclosporin A, crisaborole, empagliflozin, nitroxolone, suprofen, sulfisoxazole, methapyrilene, phentolamine, naphazoline or a pharmaceutically acceptable salt or mixture thereof. Preferred anti-DME agents are desipramine, nitroxolone, methapyriline, phentolamine, napthazoline and their pharmaceutically acceptable salts. These agents find particular use in the treatment of diabetic macular edema (DME), including inhibiting ameliorating and/or reducing the likelihood of DME in a patient or subject, as well as treating and/or reducing the likelihood of PDR, wet AMD and/or RVO in patients or subjects in need. Other additional anti-DME agents, especially anti-VEGF agents, corticosteroid and/or insulin-like growth factor receptor inhibitor compounds, may be combined with one or more of the above anti-DME agents for the treatment of DME as well as PDR, wet AMD and/or RVO.

The term “additional anti-DME agent” is used to describe compounds with activity as agents which may be used in the treatment of DME, PDR, wet-AMD and/or RVO with albeit with limited success. These additional anti-DME agents include, for example, anti-VEGF agents such as ranibizumab, aflibercept, bevacizumab, faricimab, sorafenib, dasatinib, sunitinib, nilotinib and pazopanib, corticosteroids such as dexamethasone, bethamethasone, fluocinolone acetonide, prednisone, prednisolone, methylprednisolone and triamcinolone, or an insulin-like growth factor receptor inhibitor such as figitumumab (CP-751871), ganitumab (AMG-479), cixutumumab (IMC-A12), dalotuzumab (MK-0648), R1507, Sch-717454, BMS-754807, linsitinib (OSI-906), MEDI-573 and BI836845). One or more of these anti-VEGF, corticosteroid and/or insulin-like growth factor receptor inhibitor agents may be combined with the anti-DME agent(s) as described above in providing combination therapy for the treatment of DME. Pharmaceutical compositions comprising combinations of an effective amount of at least one anti-DME agent and at least one additional anti-DME agent (e.g. at least one anti-VEGF agent, corticosteroid and/or insulin-like growth factor receptor inhibitor as identified herein), optionally in combination with at least one pharmaceutically acceptable carrier, additive and/or excipient represent additional embodiments of the present invention.

The term “diagnosing or identifying” a patient with diabetic macular edema (DME), proliferative diabetic retinopathy (PDR), wet (exudative) age-related macular degeneration (wet AMD) and/or retinal vein occlusion (RVO) or at risk for one of more of these diseases is used to describe the diagnostic or identification steps that are used by practitioners to establish that a patient or subject has one or more of these disease states, especially diabetic macular edema or is at risk for one or more of these disease states, especially diabetic macular edema such that therapy with an anti-DME agent as described above, alone or in combination with an additional anti-DME agent such as an anti-VEGF agent, a corticosteroid, or an insulin-like growth factor receptor inhibitor may be used to treat the patient.

The diagnosis of diabetic macular edema may be performed using any method which is known in the art to the practitioner and does not have to be formal, in some instances, depending on the level of skill and experience of the diagnostician or medical practitioner.

A preferred method of diagnosis of the standard diagnosis protocol recommended by the American Academy of Ophthalmology.
1. Complete Ophthalmic Examination: As part of the exam, slit lamp biomicroscopy is done to detect changes in the retina and macula of diabetic patients. Stereoscopic examination reveals retinal hemorrhages (type and location) and the presence of clinically significant macular edema (CSME) involving the fovea or in the surrounding area within 1 disc diameter. This first step may be sufficient to identify macular edema.
2. Ancillary testing and imaging is often utilized in addition to the complete ophthalmic examination described above. This ancillary testing includes:
   i. Digital mydriatic retinal photography normal or wide field is done to record the presence of retinal changes.
   ii. Optical Coherence Tomography (OCT) is done to further detect thickening, and structural changes (intraretinal fluid, cystoid macular edema, subretinal fluid), and to determine whether thickening is involving the center of vision or not.
   iii. Fluorescein angiography (FA): In selected cases, FA is also done to confirm the dynamic leakage from retinal capillaries and microaneurysms in the macular area, and also to detect capillary nonperfusion and macular ischemia.

3. Intravitreal anti-VEGF injections: These are done only when there is center-involving macular edema with vision of 20/30 or worse. If the vision is 20/20-20/25, patients are observed without any interventions of anti-VEGF injections as per the DRCR Protocol V. So, it is critical to have the OCT imaging done to determine whether the edema is center-involving or not.

A positive diagnosis that a patient has macular edema or is at risk for macular edema is made by a medical practitioner or diagnostician based upon one or more of the foregoing steps which are presented above and the analysis of the data which is collected. Once diagnosis (of illness or substantial risk) is affirmed, the patient is treated pursuant to the present invention.

The above-referenced diagnosis can be used to distinguish diabetic retinopathy from diabetic macular edema.

In the case of diabetic retinopathy (DR), this is diagnosed by slit-lamp biomicroscopy and indirect ophthalmoscopy. The stages of DR can vary from mild non-proliferative DR (few microaneurysms), moderate non-proliferative DR (dot & blot hemorrhages) to severe non-proliferative DR and proliferative diabetic retinopathy (with new vessels). In many instances, DR may lead to Proliferative diabetic retinopathy (PDR) and/or DME. In embodiments a patient may be diagnosed with DME, PDR, wet AMD and/or RVO with neovascularization or DME and PDR in combination. Regardless of the diagnosis of the patient, if DME, PDR, wet AMD and/or RVO with neovascularization is diagnosed administration of compounds as described herein represents a viable strategy for treatment.

Diabetic Macular Edema (DME) occurs in a small subset of patients with DR and can cause vision loss. The diagnosis is confirmed by slit-lamp biomicroscopy stereoscopic macular exam with macular lens and then by OCT exam to find out the severity and location of edema as discussed above. If the edema is found in the center of vision, it can cause significant vision loss. Thus, DME and/or PDR occurs in some patients with DR. In embodiments, some patients with DME are free from proliferative diabetic retinopathy (i.e. proliferative diabetic retinopathy is not also diagnosed). In other embodiments, some patients with PDR are free from DME. In still other embodiments, some patients with DME are also diagnosed with PDR. In type 1 diabetes, 100% of patients develop some sort of retinopathy (hemorrhages, microaneurysms) by 15 years, but 20% of them develop diabetic macular edema. In type 2 diabetes, 80% of patients will develop some retinopathy by 15 years, but 25% usually develop DME.

The compounds used in the methods of treatment of the present invention may be formulated in a conventional manner using one or more pharmaceutically acceptable carriers. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compounds used in the methods of treatment of the present invention may be administered orally, parenterally, by inhalation spray, topically, rectally, nasally, buccally, vaginally or via an implanted reservoir. The term “parenteral” as used herein includes subcutaneous, intravenous, intraocular, intravitreal, intraconjunctival, subconjunctival, intramuscular, intra-articular, intra-synovial, intrasternal, intrathecal, intrahepatic, intralesional and intracranial injection or infusion techniques. Preferably, the compositions are administered orally, intravenously or by intraocular administration, including intravitreal administration and intraocular implant administration. Preferred routes of administration include oral administration, intraocular administration, intraocular implant administration, intravitreal administration intraconjunctival administration, subconjunctival administration or topically (via eye drops).

Sterile injectable forms of the compounds used in the methods of treatment of the invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed, including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions used in the methods of treatment of this invention may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions used in the methods of treatment of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compounds used in the methods of treatment of this invention may also be administered topically, especially when the target of treatment includes areas or organs readily accessible by topical application. Suitable topical formulations are readily prepared for each of these areas or organs.

Topical application also can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions used in the methods of treatment of this invention may also be administered by nasal aerosol or by inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of compounds used in the methods of treatment of the instant invention that may be combined with the carrier materials to produce a single dosage form will vary depending upon the host treated, the particular mode of administration. Preferably, the compositions should be formulated so that a therapeutically effective dosage of between about 0.1 to about 300 mg/kg, 0.1 and 100 mg/kg, about 1 to about 50 mg/kg, about 1 to about 30 mg/kg, about 1 to about 25 mg/kg, about 1 to about 20 mg/kg, about 1 to about 15 mg/kg, about 2.5 to about 10 mg/kg of patient/day of the active agent(s) can be administered to a patient receiving these compositions. Preferably, pharmaceutical compositions in dosage form according to the present invention comprise a therapeutically effective amount of less than 1 mg of active agent, at least 1-10 mg of active agent, at least 25 mg of active agent(s), at least 50 mg of active agent(s), at least 60 mg of active agent(s), at least 75 mg of active agent(s), at least 100 mg of active agent(s), at least 150 mg of active agent(s), at least 200 mg of active agent(s), at least 250 mg of active agent(s), at least 300 mg of active agent(s), about 350 mg of active agent(s), about 400 mg of active agent(s), about 500 mg of active agent(s), about 750 mg of active agent(s), about 1 g (1000 mg) of active agent(s), about 2 g (2000 mg), about 3 g (3000 mg) and above of active agent alone or in combination with a therapeutically effective amount of an additional anti-DME agent. The figures which are presented in Appendices A and B may provide guidance in providing a dosage range for administration of compounds, agents and compositions according to the present invention.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

Administration of the active compound may range from continuous (intravenous drip) to several oral or topical administrations per day (for example, B.I.D. or Q.I.D.) and may include oral, topical, parenteral, intraocular, intravitreal, intraconjunctival, subconjunctival, intramuscular, intravenous, sub-cutaneous, intraocular implant, transdermal (which may include a penetration enhancement agent), buccal and suppository administration, among other routes of administration. Enteric coated oral tablets may also be used to enhance bioavailability of the compounds from an oral route of administration. The most effective dosage form will depend upon the pharmacokinetics of the particular agent chosen as well as the severity of disease in the patient. Oral dosage forms are particularly preferred, because of case of administration and prospective favorable patient compliance.

To prepare the pharmaceutical compositions according to the present invention, a therapeutically effective amount of one or more of the compounds according to the present invention is preferably intimately admixed with a pharmaceutically acceptable carrier according to conventional pharmaceutical compounding techniques to produce a dose. A carrier may take a wide variety of forms depending on the form of preparation desired for administration, e.g., oral or parenteral. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavoring agents, preservatives, colouring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, the tablets or capsules may be enteric-coated or sustained release by standard techniques. The use of these dosage forms may significantly the bioavailability of the compounds in the patient.

For parenteral formulations, the carrier will usually comprise sterile water or aqueous sodium chloride solution, though other ingredients, including those which aid dispersion, also may be included. Of course, where sterile water is to be used and maintained as sterile, the compositions and carriers must also be sterilized. Injectable suspensions may also be prepared, in which case appropriate liquid carriers, suspending agents and the like may be employed.

Liposomal suspensions (including liposomes targeted to viral antigens) may also be prepared by conventional methods to produce pharmaceutically acceptable carriers. This may be appropriate for the delivery of compounds according to the present invention.

The present invention also relates to the use of pharmaceutical compositions in an oral dosage form comprising therapeutically effective amounts of an anti-DME compound according to the present invention in combination with an additional anti-DME as described herein, optionally in combination with a pharmaceutically acceptable carrier, additive or excipient. Compositions for oral administration include powders or granules, suspensions or solutions in water or non-aqueous media, sachets, capsules or tablets. Thickeners, diluents, flavorings, dispersing aids, emulsifiers or binders may be desirable.

The pharmaceutical compositions of the invention are safe and effective for use in the therapeutic methods according to the present invention. Although the dosage of the compounds used in the methods of treatment of the invention may vary depending on the type of active substance administered as well as the nature (size, weight, etc.) of the subject to be diagnosed, the composition is administered in an amount effective for treating diabetic macular edema in a patient identified with that disease or identified with a high risk for that disease. For example, the composition is preferably administered such that the active ingredient can be given to a human adult in a dose of at least about 25 mg, at least about 50 mg, at least about 60 mg, at least about 75 mg., at least about 100 mg, at least about 150 mg, at least about 200 mg, at least about 250 mg, at least about 300 mg, at least about 350 mg, at least about 400 mg, at least about 500 mg, at least about 750 mg, at least about 1000 mg or more, and given in a single dose, including sustained or controlled release dosages once daily.

The form of the pharmaceutical composition of the invention such as a solution, suspension, droplet etc. may be suitably selected according to the type of substance to be administered.

### EXAMPLES

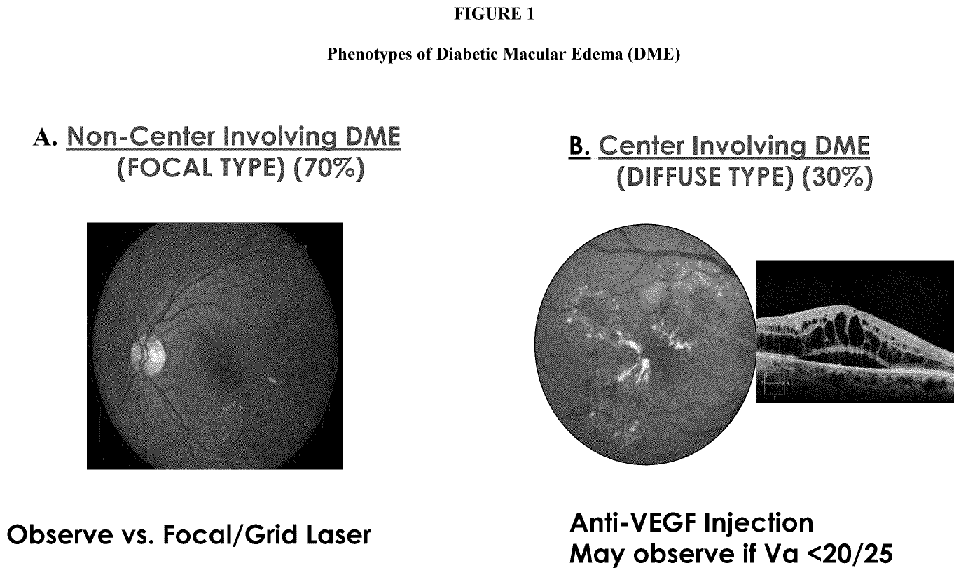

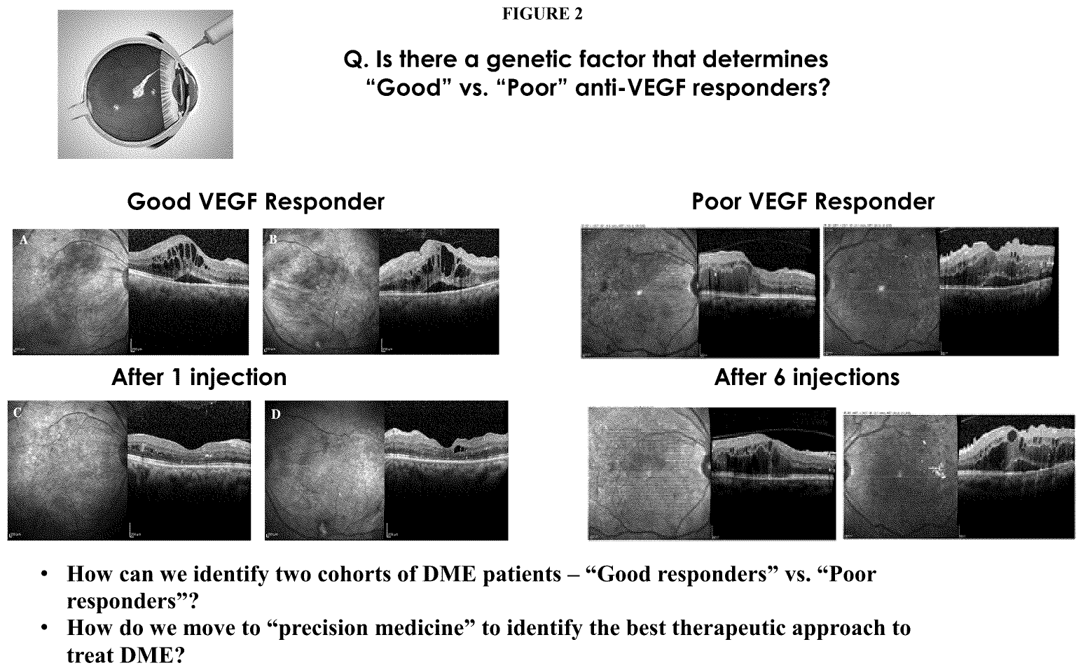

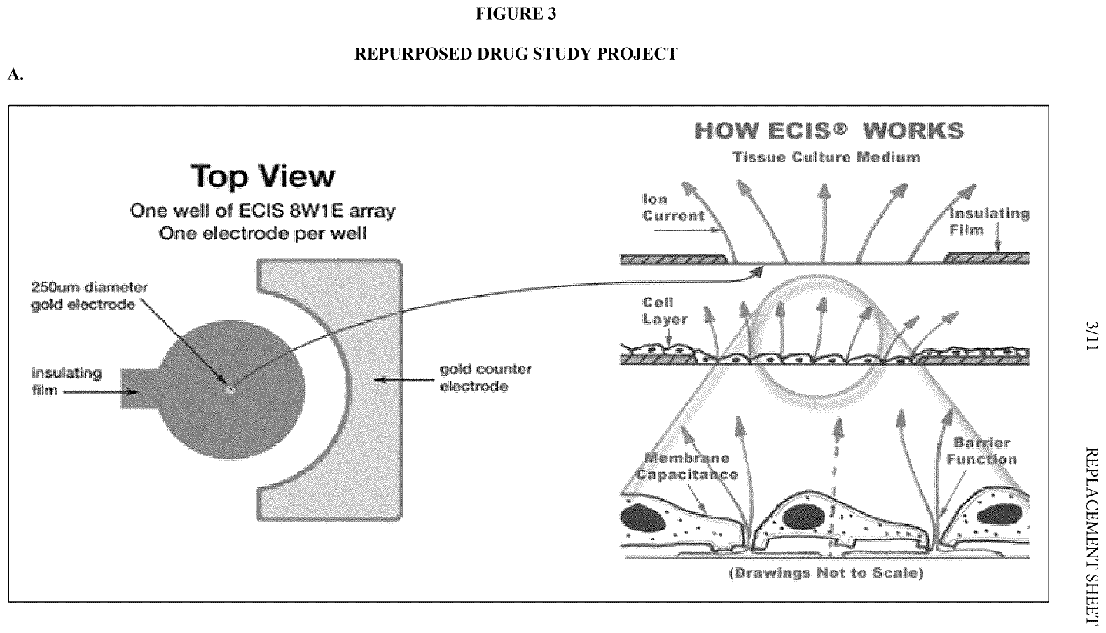

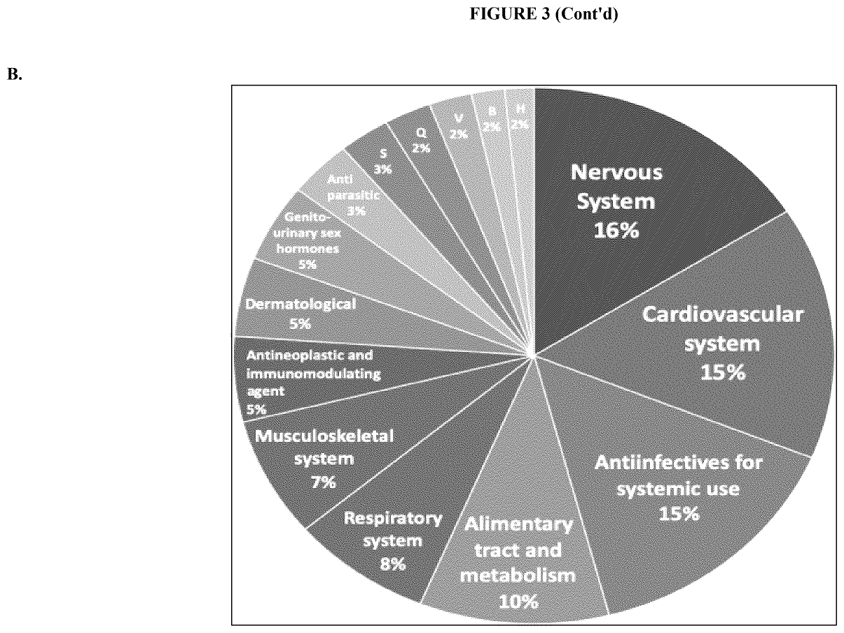

As shown in several large clinical trials, there is a need for developing novel drugs that can treat increased vascular permeability as seen in DME (Brown et al., 2013; Elman et al., 2012; Korobelnik et al., 2014). Most of the current efforts on anti-diabetic retinopathy drug discovery focus on neutralizing VEGF (FIGS. 1 & 2). Only a few drugs have been developed to block the inflammatory cascade at the initial stage when immune cells along with activated microglia infiltrate the retina. Therefore, a critical gap in anti-diabetic retinopathy drug discovery is the lack of drugs rescuing immune cell function in a diabetic condition. We checked the likelihood of being able to use a small molecule chemical library to check the efficacy on the functionally promising candidates and used a high throughput screening (HTS) assay platform to check the in vitro permeability in human retinal endothelial cells with the ECIS system (FIG. 3). The identified chemical probes can be directly used for anti-diabetic retinopathy drug development or serve to discover novel disease targets for future drug discovery.

### Repurposing of Drugs

Repurposing (also called repositioning or reprofiling or re-tasking) of a drug includes use of drugs already approved by regulatory agencies such as the FDA, the European Medicines Agency (EMA), the Medicines and Healthcare Products Regulatory Agency (MHRA), and others. Given the lengthy duration, cost and manpower involved in drug development which is usually 10-15 years and many million to billon dollars investment, repurposing is a suitable approach because of the shortened route to hit the market for the patients (Parvathaneni et al., 2019; Pushpakom et al., 2019). With an urgent need and potential for targeting and developing new therapeutics for DR, we used the expertise of Dr. Sklar from the UNM-Center for Molecular Discovery, an expert in high throughput screening assay development, drug repurposing and real-time analysis of molecular interactions (with expertise of Dr. Sklar, PhD, UNM Center for Molecular Discovery). This assay and the concept of repurposing has been molded to check the biological activity in different diseases and molecular functions like efflux profiling and antimicrobial development for resistance nodulation cell division (RND) model systems (Haynes et al., 2018), Hantavirus cell entry in hemorrhagic fever with renal syndrome (HFRS) Rho GTPase activity (Palsuledesai et al., 2018), and integrin activation (Sambrano et al., 2018).

### Results

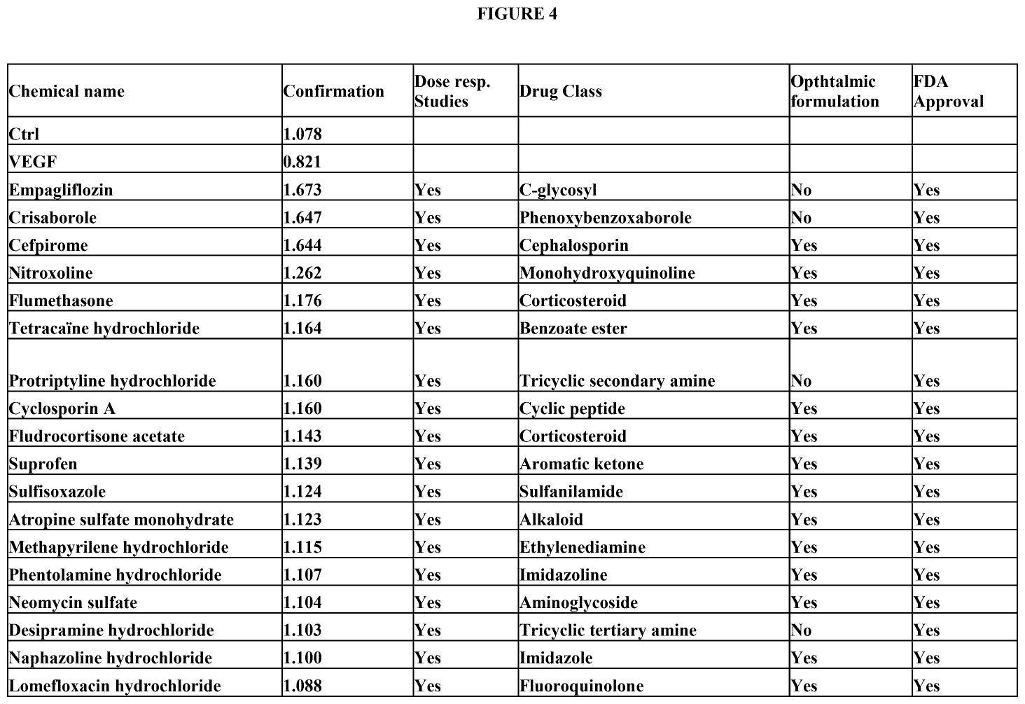

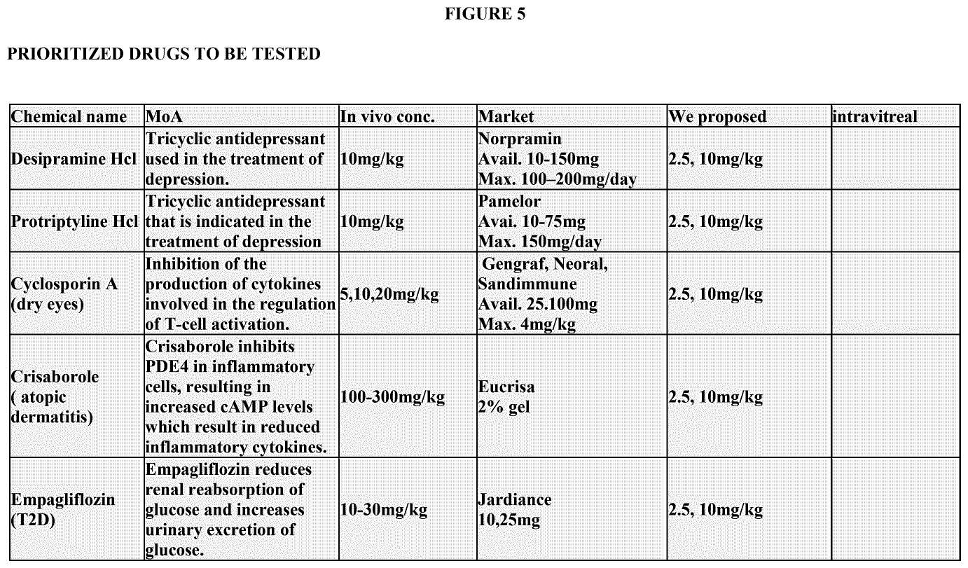

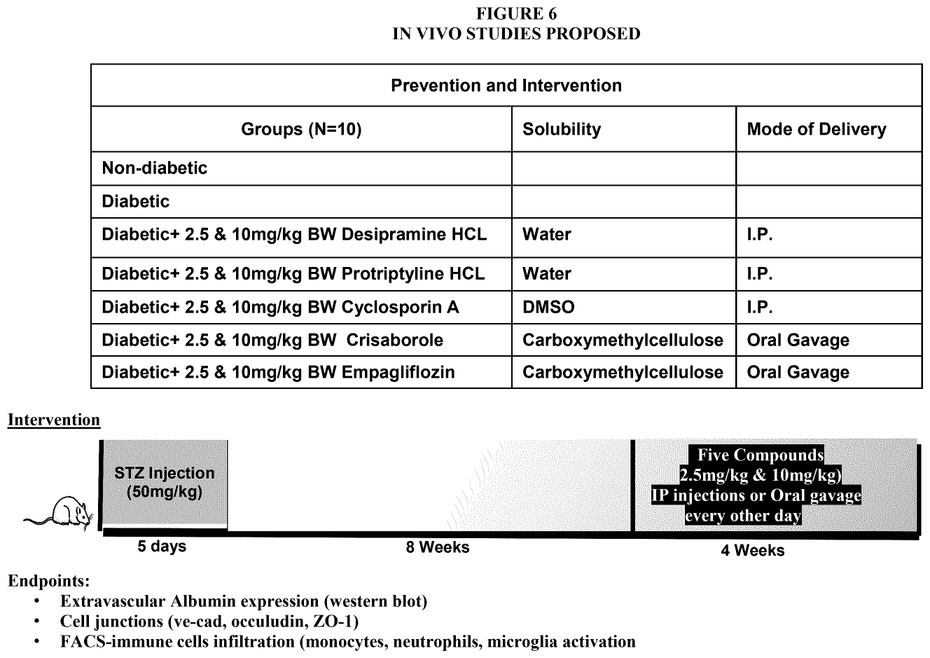

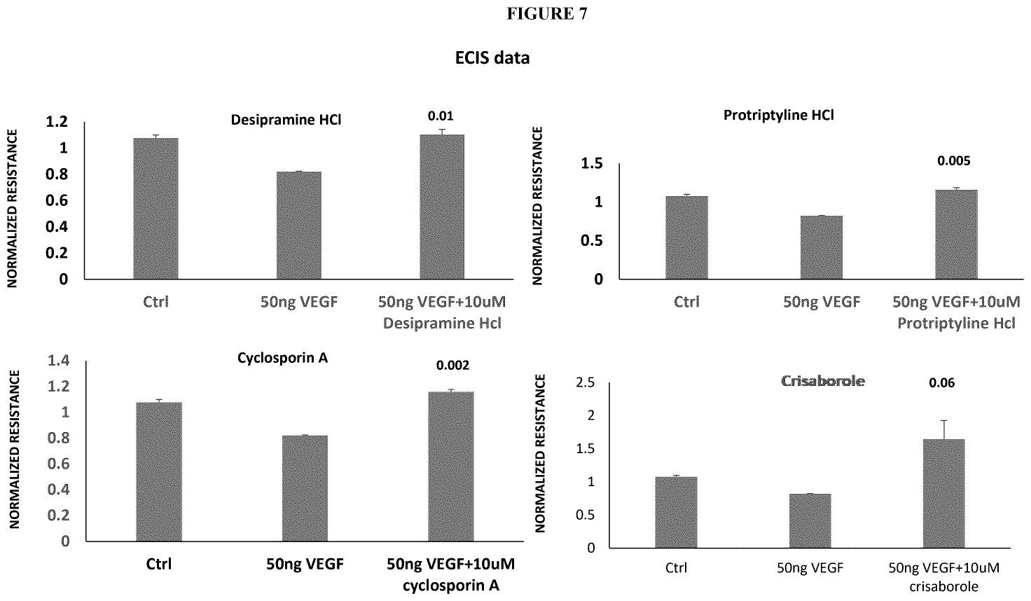

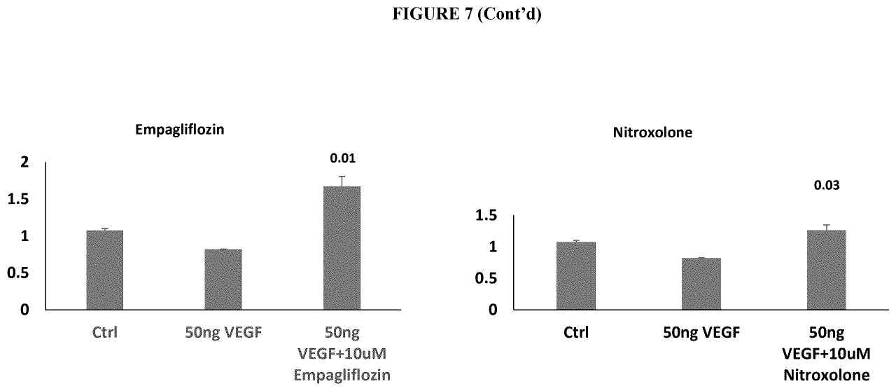

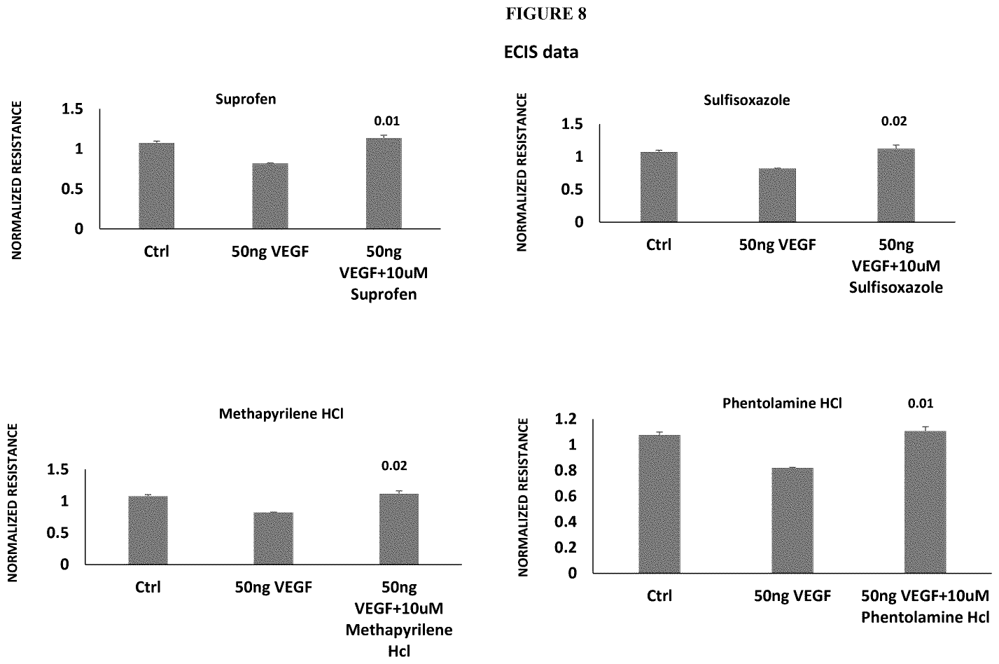

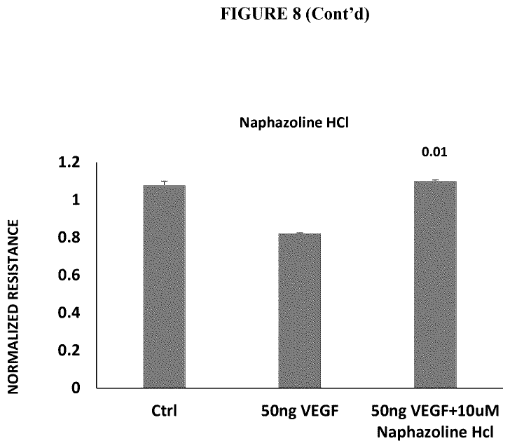

The inventors used the Prestwick chemical library compounds, which consists of a total of 1520 library compounds to check their effectiveness to decrease permeability in HRECs treated with VEGF (FIG. 4). All the available 1520 drugs were screened for toxicity using MTT (3-(4,5-dimethylthiazol-2-yl)-2,5-diphenyltetrazolium bromide) assay with the recommended concentration of 10 uM. We kept a cutoff range of >75% viability, and 209 compounds showed toxicity. The in vitro permeability or Electrical Cell-Substrate Impedance Sensing (ECIS) assay was carried out using Applied biophysics Z theta instrument, using 96W10idf PET plates. The HRECs were first seeded on the plates and the cells were allowed to grow until the desired resistance was achieved (800-1200V), this usually takes 15-18 hrs. After that the cells were treated with 50 ng/ml Rec. VEGF and the compounds at 10 uM concentration. At the end of 24 hrs, the resistance was calculated (an increase in resistance implies decreased permeability). Out of the 150 compounds tested out of which five compounds showed significantly increased resistance (FIGS. 5-8).

### Conclusion

The top five compounds as listed have shown significant restoration of the endothelial cell barrier function and may thus have enormous translational implication in retinal diseases where the endothelial cell permeability is compromised resulting in alteration of the blood-retinal barrier.

1. A method of treating diabetic macular edema (DME), proliferative diabetic retinopathy (PDR), wet (exudative) age-related macular degeneration (wet AMD) and/or retinal vein occlusion (RVO) in a patient or subject in need comprising administering to said patient or subject an effective amount of at least one anti-DME agent selected from the group consisting of desipramine, protriptyline, cyclosporin A, crisaborole, empagliflozin, nitroxolone, suprofen, sulfisoxazole, methapyrilene, phentolamine, naphazoline or a pharmaceutically acceptable salt thereof.

2. The method according to claim 1 wherein said anti-DME agent is at least one agent selected from the group consisting of desipramine, nitroxolone, methapyriline, phentolamine, napthazoline or a pharmaceutically acceptable salt thereof.

3. The method according to claim 1 or 2 wherein said anti-DME agent is despiramine or a pharmaceutically acceptable salt thereof.

4. The method according to claim 1 or 2 wherein said anti-DME agent is co-administered with at least one additional anti-DME agent.

5. The method according to claim 4 wherein said additional anti-DME agent is at least one agent selected from the group consisting of an anti-VEGF agent, a corticosteroid and an insulin-like growth factor.

6. The method according to claim 5 wherein said anti-VEGF agent is at least one agent selected from the group consisting of ranibizumab, aflibercept, bevacizumab, sorafenib, dasatinib, sunitinib, nilotinib, pazopanib and a pharmaceutically acceptable salt thereof.

7. The method according to claim 5 wherein said corticosteroid is at least one agent selected from the group consisting of dexamethasone, bethamethasone, prednisone, prednisolone, methylprednisolone, triamcinolone and a pharmaceutically acceptable salt thereof.

8. The method according to claim 5 wherein said an insulin-like growth factor receptor inhibitor is selected from the group consisting of figitumumab (CP-751871), ganitumab (AMG-479), cixutumumab (IMC-A12), dalotuzumab (MK-0648), R1507, Sch-717454, BMS-754807, linsitinib (OSI-906), MEDI-573, BI836845) and a pharmaceutically acceptable salt thereof.

9. The method according to any one of claims 1-8 which treats DME in the patient or subject in need.

10. The method according to any one of claim 1-8 which treats PDR in the patient or subject in need.

11. The method according to any one of claims 1-8 which treats wet AMD in the patient or subject in need.

12. The method according to any one of claims 1-8 which treats RVO with neovascularization in the patient or subject in need.

13. The method according to any one of claims 1-8 which treats both DME and PDR in the patient or subject in need.

14. A method of treating diabetic macular edema (DME), proliferative diabetic retinopathy (PDR), wet (exudative) age-related macular degeneration (wet AMD) and/or retinal vein occlusion (RVO) in a patient or subject in need comprising a) diagnosing or identifying that a patient or subject has DME, PDR, wet AMD and/or RVO; and b) administering to said patient or subject an effective amount of at least one anti-DME agent selected from the group consisting of desipramine, protriptyline, cyclosporin A, crisaborole, empagliflozin, nitroxolone, suprofen, sulfisoxazole, methapyrilene, phentolamine, naphazoline or a pharmaceutically acceptable salt thereof.

15. The method according to claim 14 wherein said anti-DME agent is at least one agent selected from the group consisting of desipramine, nitroxolone, methapyriline, phentolamine, napthazoline or a pharmaceutically acceptable salt thereof.

16. The method according to claim 14 wherein said anti-DME agent is despiramine or a pharmaceutically acceptable salt thereof.

17. The method according to any one of claims 14-16 wherein said anti-DME agent is co-administered with at least one additional anti-DME agent.

18. The method according to claim 17 wherein said additional anti-DME agent is at least one agent selected from the group consisting of an anti-VEGF agent, a corticosteroid and an insulin-like growth factor receptor inhibitor.

19. The method according to claim 18 wherein said anti-VEGF agent is at least one agent selected from the group consisting of ranibizumab, aflibercept, bevacizumab, sorafenib, dasatinib, sunitinib, nilotinib, pazopanib and a pharmaceutically acceptable salt thereof.

20. The method according to claim 18 wherein said corticosteroid is at least one agent selected from the group consisting of dexamethasone, bethamethasone, prednisone, prednisolone, methylprednisolone, triamcinolone and a pharmaceutically acceptable salt thereof.

21. The method according to claim 18 wherein said an insulin-like growth factor receptor inhibitor is selected from the group consisting of figitumumab (CP-751871), ganitumab (AMG-479), cixutumumab (IMC-A12), dalotuzumab (MK-0648), R1507, Sch-717454, BMS-754807, linsitinib (OSI-906), MEDI-573 (BI836845) and a pharmaceutically acceptable salt thereof.

22. The method according to any one of claims 14-21 wherein said patient or subject is diagnosed with DME and/or PDR.

23. The method according to any one of claims 14-22 wherein said patient or subject diagnosed with PDR is free from diabetic macular edema.

24. The method according to any one of claims 14-22 wherein said patient or subject is diagnosed with both DME and PDR simultaneously.

25. A pharmaceutical composition comprising an anti-DME agent selected from the group consisting of desipramine, protriptyline, cyclosporin A, crisaborole, empagliflozin, nitroxolone, suprofen, sulfisoxazole, methapyrilene, phentolamine, naphazoline or a pharmaceutically acceptable salt thereof, in combination with at least one anti-VEGF agent selected from the group consisting of ranibizumab, aflibercept, bevacizumab, sorafenib, dasatinib, sunitinib, nilotinib and pazopanib and their pharmaceutically acceptable salts, at least one corticosteroid selected from the group consisting of dexamethasone, bethamethasone, prednisone, prednisolone, methylprednisolone, triamcinolone and their pharmaceutically acceptable salts, and/or at least one insulin-like growth factor receptor inhibitor selected from the group consisting of figitumumab (CP-751871), ganitumab (AMG-479), cixutumumab (IMC-A12), dalotuzumab (MK-0648), R1507, Sch-717454, BMS-754807, linsitinib (OSI-906), MEDI-573 and BI836845), and their pharmaceutically acceptable salts, optionally in combination with a pharmaceutically acceptable carrier, additive or excipient.

26. The composition according to claim 25 wherein the anti-DME agent is selected from the group consisting of desipramine, nitroxolone, methapyriline, phentolamine, napthazoline and their pharmaceutically acceptable salts.

27. The composition according to claim 26 wherein the anti-DME agent is despiramine.

28. The composition according to any one of claims 25-27 which comprises at least one anti-VEFG agent.

29. The composition according to any one of claims 25-28 which comprises at least one corticosteroid.

30. The composition according to any one of claims 25-29 which comprises at least one insulin-like growth factor receptor inhibitor.
